(19) United States
(12) Patent Application Publication
(10) Pub. No.: US 2014/0369885 A1
(43) Pub. Date: Dec. 18, 2014

(54) APPARATUS AND METHOD FOR THE STERILIZATION OF CONTAINERS WITH MONITORING OF FUNCTIONS

(71) Applicant: Jochen Krueger, Hagelstadt (DE)

(72) Inventor: Jochen Krueger, Hagelstadt (DE)

(73) Assignee: KRONES AG, Neutraubling (DE)

(21) Appl. No.: 14/366,734

(22) PCT Filed: Dec. 19, 2012
(86) PCT No.: PCT/EP2012/076186

§ 371 Date: Jun. 19, 2014

(30) Foreign Application Priority Data
Dec. 19, 2011 (DE) 10 2011 056 628.7

(51) Int. Cl. A61L 2/08

(52) U.S. Cl. CPC A61L 2/087
USPC 422/22; 250/393

(57) ABSTRACT

A method is provided for operating a device for sterilising containers, wherein the containers are transported along a predetermined transport path by a transport device and during the transportation at least one wall of the containers is irradiated with radiation, in particular charge carrier radiation, by at least one first radiation device, and wherein a sensor device is also provided which detects charge carrier radiation and/or radiation caused by the charge carriers and which is arranged such that the radiation incident on the sensor device fluctuates due to the movement of the containers along the transport path, and this sensor device detects at least one characteristic radiation variation. Wherein at least one first limit value is defined and a comparison between this limit value and the radiation variation is performed. According to the method, the limit value has a value that changes over time.

The present invention relates to an apparatus and a method for the sterilization of containers, and in particular for the sterilization of containers by means of charge carriers.

In the field of processing plants and methods of producing drinks containers the sterilization of the containers (for example of bottles or even plastics material pre-forms which are shaped to form bottles) is—as well as the actual filing procedure—the central process step in an aseptic filling plant. When the containers are disinfected by means of electron beams, on the one hand the safety of the processing, i.e. the sterilization action upon each individual container is important. On the other hand, however, the safety of the operators and the personnel of the plant is also an essential aspect.

In recent years, there has also been a move towards acting upon the containers by means of charge carrier radiation, and in particular by means of electron radiation, in order to achieve sterilization in this way. When the containers are irradiated with high--energy electrons, x--ray radiation also occurs as an undesired side effect. These x-ray beams occur when the electrons are braked, for example at the titanium foil, of the exit window, but also when they strike the containers. In order to protect the person and also electronic components in the vicinity of the machine from this radiation, screens are attached which consist of a material with an adequate wall thickness. For electron beams with an amount of energy in the range of 150 keV, lead walls with a thickness in the range of from 5 to 12 mm are required (depending upon the current strength) for screening off the x-ray radiation. Alternately, however, another material, such as for example steel, can also be used, but the screening effect of steel is significantly lower, so that the wall thickness becomes correspondingly thicker.

Usually, however, the entire plant is screened off in such a way that in the case of the defined parameters of the radiation and an operation of the plant in accordance with its intended use, i.e. normal operation, a residual radiation of more than 1 μSv/h cannot occur at any point outside the plant.

Failures (relevant to safety) can also occur, however, if for example one or more of the radiators are defective or incorrectly actuated, so that an increased amount of radiation is produced.

Detectors are necessary in order to ensure this operation in accordance with the intended use, or normal operation. They can be any x-ray or radiation detectors or dosimeters which measure a specified radiation level inside or outside the machine during operation. If this measurement signal exceeds a pre-set threshold value, then the maximum residual radiation outside the machine can be exceeded, and this will lead the operating staff being put at risk. As a consequence, the plant has to be immediately switched off (emergency shutdown). If, on the other hand, a pre-set threshold value is not reached, this indicates failure or a fault on one or more radiators in the plant, with the result that the sterilization effect upon the containers is no longer ensured. In this case too, it is necessary for counter measures to be initiated.

A sterilization device with electron radiators is known from EP 2 316 495 A1. In this case an electron radiation device arranged in a stationary manner is provided, as well as a detector which is opposite the containers and which receives the electron beams. In order to be able to detect a fault of this type, threshold values are set which are above and below the radiation received in each case.

U.S. Pat. No. 7,641,851 B2 describes a method and an apparatus for the evaluation of a sterilization process. The object of this method is to monitor the sterilization of medical products. In this case a minimum sterilization dose is determined which achieves a specified desired degree of sterility. After that, a test component and a plurality of indicators are supplied to this electron beam. A radiation distribution is then reviewed on this component. This method is relatively complicated and requires a plurality of radiation detectors in order to test the effectiveness of the irradiation.

An apparatus and a method of protecting circuits during sterilization processes are also known from WO 00/71173 12.

In general, however, even in the case of different measurement methods it can happen that the measurement signal is suspended on account of a fault of the sensor or detector respectively and erroneously submits a measurement value which is supposedly in de safe range. For this reason, in safety technology, use is made of dynamic measurement techniques which, in addition to a specified measurement size, also measure and evaluate a periodic fluctuation. For this purpose, the measurement sizes are periodically modulated and the sensor has to follow these modulations.

Use can now be made of this principle in electron beam sterilization in order to monitor the plant in a purposeful manner. Not only should the plant therefore have installed outside it a sensor which ensures that the radiation level outside the plant remains below the permitted threshold value, but the detector should be positioned in such a way that in any case it measures a significant radiation signal. It is also preferable for it to be positioned at a place in which strong or in any case weakened radiation prevails. The positioning of a sensor of this type is very difficult and in no way ensures that radiation which does not reach this sensor will not escape at a different point on the machine. The diffusion of x-ray radiation from a small leak (for example a door gap or an inadequate welding seam) is substantially in a straight line and can pass by the sensor at very high strength.

In this case, however, the problem arises that the detector signals mentioned above fluctuate and even fluctuate in a desired manner. On account of the formation of threshold values in the publication mentioned above, it therefore becomes necessary for the threshold values to be at a very great distance from each other. The object of the invention is therefore to improve or make more precise this measurement of the radiation which occurs. These objects are attained by the subjects of the independent claims. Advantageous embodiments and further developments form the subject matter of the sub-claims.

In the case of a method according to the invention for the operation of an apparatus for the sterilization of containers, the containers are conveyed along a pre-set conveying path by means of a conveying device and during the conveying of these containers at least one wall of the containers is irradiated with a radiation—and in particular a charge carrier radiation—by means of at least one first radiation device. In addition, a sensor device is provided which detects charge carrier radiation or radiation produced by the charge carriers and which is arranged in such a way that the radiation striking the sensor device is subjected to fluctuations on account of the movement of the containers along the conveying path, this sensor device detecting at least one pattern characteristic of the radiation, and at least one threshold value is defined, and a comparison is made between this threshold value and the pattern.

According to the invention the threshold value has a value (in particular defined) which is variable—preferably in terms of time and/or location—or varying (in particular in working operation) resbectively.

The arrangement of the sensor device is thus deliberately chosen in such a way that a constant signal is not received by it, but the aforesaid signal fluctuates as a result of the movement of the containers. If the radiation devices move jointly with the containers it is sufficient for the sensor device to be arranged in a stationary manner, since the radiation striking the sensor device will already change on account of the varying distances between the radiation device and the sensor device.

In contrast to the prior art, therefore, a constant threshold value is not set, but the aforesaid threshold value varies. It is advantageous for the threshold value to have a pattern (in particular a time and/or location-dependent pattern) which is adapted to the characteristic pattern of the radiation measured. In this way, it is possible for the fluctuations characteristic of the pattern to be absorbed for example within the context of a learning operation and also for the threshold value to be adapted to this pattern. In the case of an advantageous embodiment the at least one sensor or detector device is arranged so as to be stationary. It would also be possible, however, for the sensor device to be moved jointly with the containers. A modulation of the threshold value which is dependent upon location and/or time is therefore proposed.

The idea is now to position the sensor device in such a way that, on account of the containers moving past, it is subjected to and evaluates a signal modulated with respect to time.

With reference to the form of the signal (intensity and time pattern) it is advantageously possible to decide in comparison with a learn signal shape whether a fault is present (for example a container is missing in the row or the emitter is emitting radiation which is too strong or too weak).

For the redundancy of the measurement and for the monitoring of the two (surface) emitters, it is also possible for a plurality of these sensors/systems to be installed in the machine, which measure and evaluate a different dynamic signal shape in each case.

For the internal sterilization of the containers, the containers are advantageously processed on a turntable. A plurality of radiation emitters are installed on this turntable and the container is moved relive to the emitter - in particular in a longitudinal direction of the container. The container can be both glass or plastics material containers and plastics material pre-forms which are capable of being shaped into plastics material containers.

The radiation source which emits radiation to be determined in this case is in this case the radiation finger itself or the titanium foil respectively and/or the plastics material pre-form or the plastics material container respectively on which the electrons are scattered.

If a sensor is now positioned at a fixed point of the plant in the vicinity of the processing turntable, then as the individual stations move past this sensor will receive a signal which is periodically modulated and the intensity of which is dependent upon the setting angle of the turntable. The time periodicity of the signal thus depends upon the distance and the speed of the radiators moving past. (rotational speed of the turntable). The shape of the periodic signal can depend upon many factors such as for example the form of the containers. It should, however, he the same with the scope of tolerances for each container during the production and processing of similar containers, in a manner comparable to a fingerprint.

Provided that all the radiators are functioning as intended, the measurement signal will move within a tolerance range in a manner dependent upon the measurement accuracy and statistical fluctuations. To this end, the system can be “learnt”, in particular by means of she threshold values described above. The shape of the signal is measured and saved during a demonstrable functioning and secure process. The measured signal shape is compared (preferably continuously) with the saved form during the subsequent production. In this case it is advantageous for a separate curve shape to be stored or plotted respectively for each individual processing station, so that in the event of a change of radiator only one curve shape has to be trained and parameterized with respect to sensitivity.

It is advantageous for at least two sensor devices to be provided which in each case detect the radiation or a pattern characteristic of the radiation. In this case it is preferable for these sensor devices to be arranged in such a way that the radiation striking them fluctuates on account of the movement, of the containers. In this case at least two sensor devices of this type are preferably based upon different measurement principles. In this way, a redundancy is achieved for the measurement.

In the case of an advantageous method the threshold value is adapted, as mentioned above, to the time pattern characteristic of the radiation.

In the case of a further advantageous method she sensor device is arranged in such a way that at least portions of the containers and/or the holding devices holding the containers are conveyed between the radiation device and the sensor device. This variant is particularly relevant when both the radiation device and the sensor device are arranged so as to be stationary. In this case the fluctuation of the radiation impinging on the sensor device is also caused in particular by the varying screening of the radiation by the containers or the holding devices thereof.

In the case of a further advantageous method the at least one radiation device is moved jointly with the containers. In this case it is possible for the radiation device likewise to be on the same carrier, for example the conveying device, which also conveys the containers. In the case of a further advantageous method at least one portion of the radiation device is inserted into an inner space of the containers. As is known per se from the prior art, the radiation device can be designed in this case in the form of a radiation finger which is inserted through the aperture of the containers into the interior of the containers. Such a procedure is known for example from 1 982 921 A1, the subject matter of which is hereby also made the subject matter of the present application by reference in its entirety.

It is advantageous for at least one external, surface and at least one internal surface of the containers to he acted upon with electron radiation. It is advantageous for the inner walls of the containers to be irradiated with a plurality of radiation devices. In this way, a plurality of the radiation fingers mentioned above can be used for example, which dip into the different containers in order to stabilize them.

In the case of a further advantageous method it is possible to determine by the comparison between the threshold value and the (measured) pattern of the radiation whether the irradiation of the containers meets pre-set criteria. It is pointed out here that, although particles are involved physically in this case, even the electrons themselves are also detected as radiation.

In the case of a further advantageous method it is also possible for a second threshold value to be defined and for a comparison to be made between this second threshold value and the pattern. In this way, a maximum threshold value for example can be defined, as well as also a minimum threshold value, and it is then possible to determine in this case that the measured pattern must always be in a range formed by these two threshold values. It is advantageous for the second threshold value also to have a pattern which is variable with respect to time and in a particularly preferred manner likewise to be adapted to the time pattern characteristic of the radiation. In the case of a further advantageous method a defined radiation device can be identified in particular from a comparison between the pattern and the threshold value. It is possible in this case, in particular, for a radiation device of this type to be identified which is responsible for the error. In this case, use can be made of the rotational position of tho conveying wheel or the conveying device respectively.

For the internal sterilization of the containers, the containers are processed on a turntable. A plurality of radiation emitters are installed on this turntable, and the container is moved relative to the emitter.

In the event of a fault of one of the radiators the measurement signal leaves she tolerance range and a fault is reported. On account of the angular dependence of the measurement signal this fault can also be assigned to a specified station.

In this way, an aged or defective radiator can be identified on account of failing to reach the tolerance threshold in the measurement signal. The container processed on this station can be separated out. In addition, a corresponding report can be made to the machine operator.

In the event of a radiation being significantly increased, an alarm can be triggered and the plant can be put into a safe state.

The amplitude of this signal swing can be significantly amplified if the sensor registers by shading or screening only radiation which comes from a specified direction. Depending upon the setting of the turntable, the detector then perceives a maximum signal when a radiation finger is directly in front of it. If a finger is not in front of the detector, the latter perceives only a diffuse background from scattered radiation.

It is also important, however, to measure this diffuse background, and it should not be too weak. It has to be stationary higher than if no radiation is present. As a result, it is detected that radiation is still present in the machine. The screening around the detector should not be too strong.

A further possibility for the processing of the containers is to install one or more radiation emitters on the plant (and not on The turntable) in a fixed manner. This is employed specially for the external sterilization of the containers, but i can also be used for the internal sterilization.

The containers move past this emitter installed in a fixed manner and are irradiated during this. In this case the sensor can be installed on the opposite side of the emitter, so that it is shaded by the containers to be processed. As a result of the containers moving past, a periodic signal modulated with respect to time is generated on the detector in this way.

The sensor device can also be installed below the pre-forms on the base of a channel, inside which the containers are conveyed, and by means of suitable shading (for example a long pipe adjoining the sensor device) it can look out only directed upwards towards the path of the pro-forms. In this way, a maximum modulation stroke should again be achieved.

With reference to the shape of the signal (intensity and duration) it is possible to decide—in comparison with a learned signal shape - whether a fault is present (for example that a container is missing in the row or that the emitter is transmitting radiation which is too strong or too weak).

It is also possible for a plurality of these sensors / systems to be installed in the machine, which measure and evaluate a different dynamic signal shape in each case.

The sources for the x-ray radiation are likewise in this case the titanium foil of the emitter and the containers, in particular pre-forms, and optionally also the holding means thereof which lead through the electron beam.

It is preferable to decide on the basis of the signal shape whether a process on an individual container is excessive or whether the radiation exceeds a dangerous limit.

The radiation detector devices mentioned can be in the form of a dosimeter which determines the electron beam or the electron flow respectively, but it would also be possible to use x-ray detectors which measure the x-ray radiation resulting from the electron radiation.

The present invention further relates to an apparatus for the sterilization of containers, which has a conveying device which conveys the containers along a pre-set conveying path, and at least one first radiation device which acts upon at least one wall of the containers with a radiation, and in particular a charge carrier radiation. In addition, the apparatus has a sensor device which detects charge carrier radiation and/or radiation produced by the charge carriers and preferably emits a signal which is characteristic of at least one characteristic property of the radiation striking the sensor device (for example the intensity thereof) and which is arranged in such a way that the radiation striking the sensor device is subjected to fluctuations on account of the movement of the containers along the conveying path.

In this case the sensor device detects at least one value pattern characteristic of the radiation and, in addition, a comparator device is provided which compares the characteristic value pattern with at least one first threshold value. According to the invention the threshold value has a variable value (in particular defined) with respect to location and/or time). It is preferable for the sensor device to detect radiation which results from the movement of the charge carriers, in particular x-ray radiation. It would also be possible, however, for the sensor device to detect electron radiation in a direct manner.

It is advantageous for the conveying device to be a wheel on which are also arranged a plurality of holding devices which hold the containers during the conveying thereof. These holding devices can be for example gripping clamps which grip the containers at a pre-set position, for example below the carrying ring thereof.

In the case of a further advantageous embodiment the apparatus has a plurality of radiation devices which irradiate different containers in each case. It is advantageous in this case for the radiation devices to be inserted into the containers. In addition, it is also advantageous for radiation devices, in particular electron radiators, to be provided which irradiation outer wails of tho containers or act upon them with charge carriers respectively. These irradiation devices are advantageously arranged so as to be stationary.

In the case of a further advantageous embodiment movement devices are provided which move, the containers relative to the radiation devices, so that the radiation devices are inserted into the containers at least in part by way of the aperture of the latter. It is advantageous in this case for the movement devices to move the containers, the movement direction advantageously being at a right angle to a conveying direction of the containers along the conveying path thereof.

In the case of a further advantageous embodiment the apparatus also has a position detection device which detects a position of the conveying device. This can be for example a rotary encoder. These rotary encoder sionals can be emitted in order to be able to determine a detected defective radiation device in this way in the case of a plurality of radiation devices.

In addition, the apparatus advantageously has a clean room inside which the sterilization is carried out. In this case it would be possible for both the external sterilization and the internal sterilization of the containers to be carried out inside this clean room, but it would also be possible for one of the two sterilization procedures to be carried out outside the clean room.

In the case of a further advantageous embodiment the conveying device has for the conveying at least one carrier which is rotatable about an axis of rotation and on which the holding devices for holding the containers are preferably arranged. It is advantageous for the conveying device to have at least two rotatable carriers, in which case the internal sterilization is carried out during the conveying of the containers on the first rotatable carrier and the external sterilization of the containers is carried out during the conveying of the containers on the second rotatable carrier.

In the case of a further embodiment the apparatus also has at least one screening device for screening off toe radiation which occurs, such as, in particular, x-ray radiation.

Further advantages and embodiments may be seen in the accompanying drawings. In the drawings

FIG. 1 is a partial view of an apparatus according to the invention in a first embodiment;

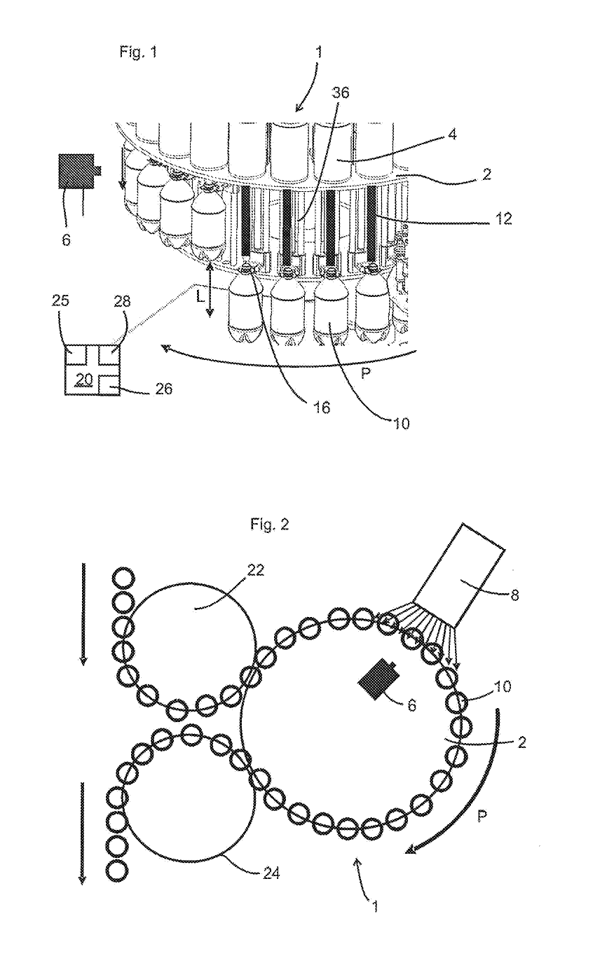

FIG. 2 is a further view of an apparatus according to the invention in a second embodiment;

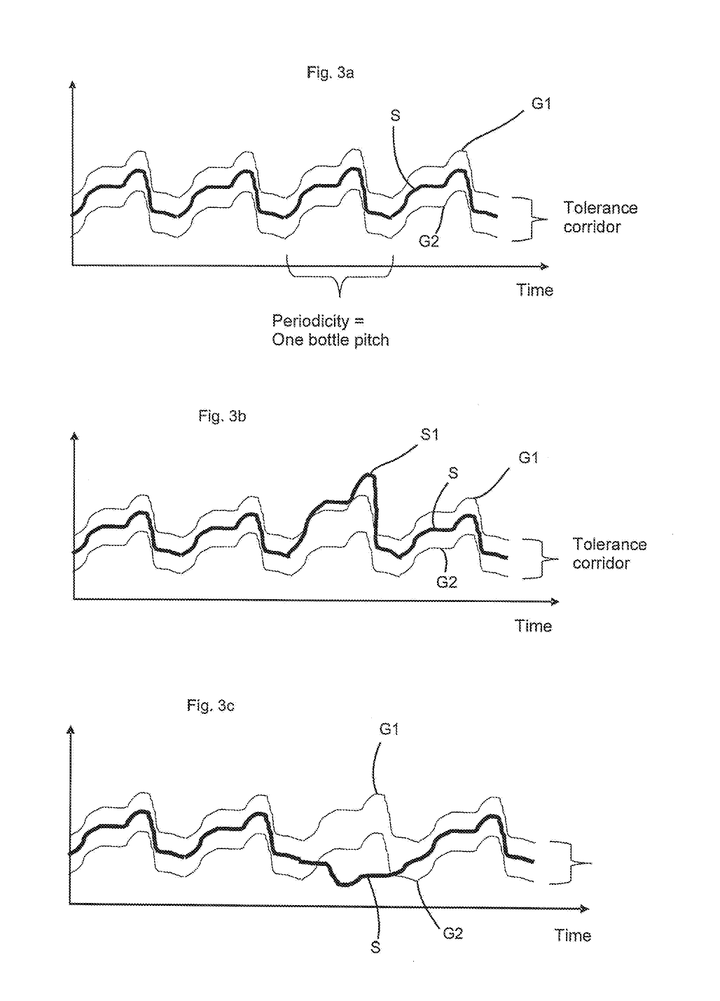

FIGS. 3a-c are three illustrations to show a sensor signal and the associated threshold values;

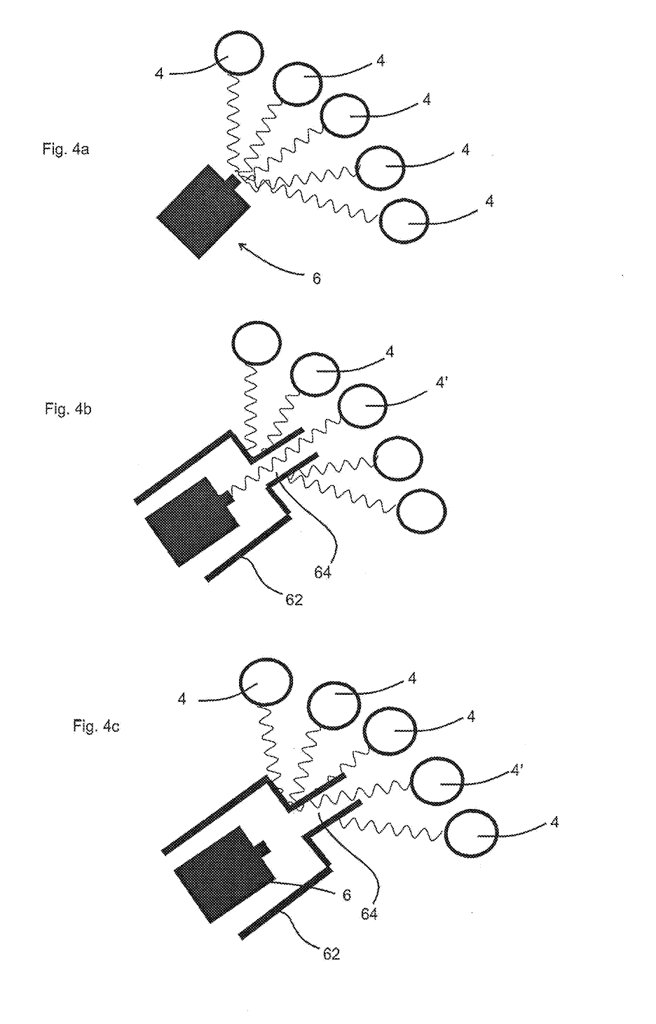

FIGS. 4a-c are three illustrations to show a possible sensor arrangement;

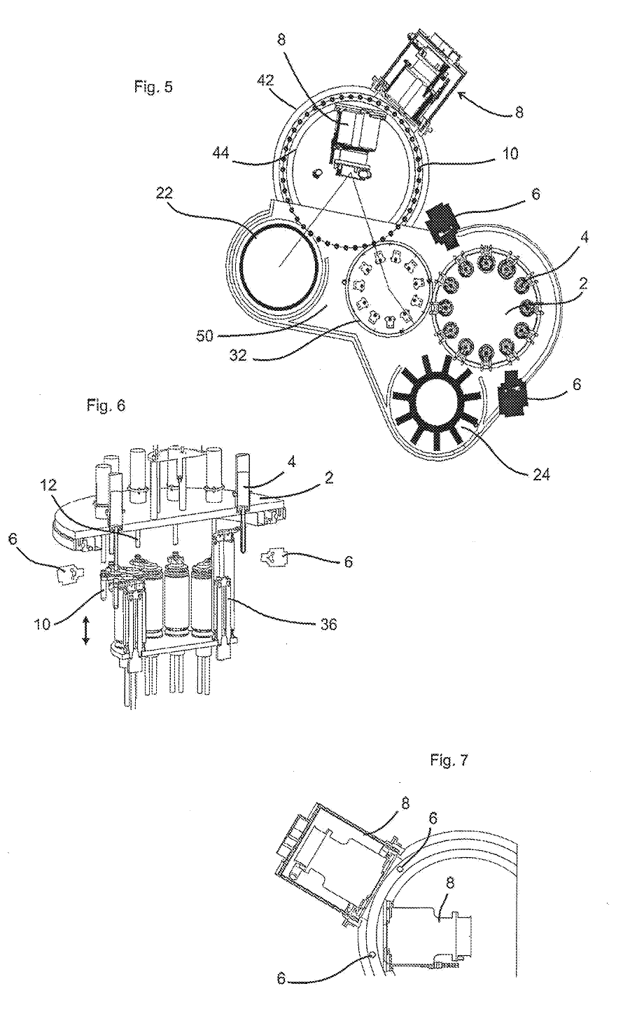

FIG. 5 is an illustration of a plant according to the invention for the sterilization of containers;

FIG. 6 is an illustration to show the arrangement of the sensor devices during the internal irradiation; and

FIG. 7 is an illustration to show the arrangement of the sensor devices during the external irradiation.

FIG. 1 is a partial view of an apparatus 1 according to the invention for the sterilization of containers 10. In this case a conveying device 2 is provided, such as for example a conveying wheel, on which are arranged a plurality of holding devices 16 which grip the containers 10 below the carrier ring thereof in each case. These holding devices 16 are also movable in this case along the longitudinal direction L thereof with a reciprocating movement, so that the radiation fingers 12, which form a component part of the radiation devices designated 4 as a whole, are capable of being inserted into the containers. In this way, the radiation devices 4 are also arranged in this case on the conveying wheel or the conveying device 2 respectively. At the lower end of the radiation fingers the latter have an exit window (not shown) through which the charge carriers can issue and enter the interior of the containers 10. The reference letter P refers to the conveying path of the containers 10 or the rotational direction of the conveying device 2 respectively.

The reference number 6 designates a radiation sensor which in this case receives radiation, in this case in particular x-ray radiation, which occurs on account of the radiation emitter (or the electrons emitted by the latter respectively). This radiation detector device 6 is arranged so as to be stationary in each case and is thus arranged in such a way that at least one wall, of the individual containers 10 is arranged at least for a time between the radiation devices 4, or more precisely between the radiation fingers 12 and the radiation detector device 6.

The reference number 20 designates a control device for controlling the individual radiation devices and for example also the reciprocating movements of the containers and the reference number 26 designates (roughly diagrammatically and not true to position) a rotary encoder, with the aid of which a rotary position of the conveying wheel 2 can also be detected.

The reference number 28 refers to a comparator device which compares the signals received by the radiation detector device 6 with threshold values stored in a memory device 25.

It would be possible in this case for the radiation data first to be received in the framework of a learning operation or a radiation pattern typical of the apparatus

FIG. 2 shows a further embodiment of an apparatus according to the invention. In the case of this embodiment a radiation device 4 arranged so as to be stationary is provided, which in this case irradiates the outer walls of the containers 10 with electron radiation. The reference number 22 refers to a supply wheel which supplies the containers 10 to the apparatus 1, and the reference number 24 designates a removal wheel which removes the containers sterilized in the outer region from the apparatus. The reference letter P in turn designates in this case the conveying -oath of the containers. In this way, the radiation device is fixed in this case and the containers 10 moves it. On account of the shading effect of the containers between the radiation device and the radiation detection device 6 a signal modulated with respect to time is produced on this radiation detector device.

FIGS. 3a to c illustrate the signals received in each case or the formation of threshold values respectively. In this case the reference letter S refers in all three illustrations in each case to the control signal received. This fluctuates periodically in this case in the two embodiments shown in FIGS. 1 and 2, since in the case of the embodiment shown in FIG. 1 the radiation devices themselves move past the radiation detector device and in the case of the embodiment shown in FIG. 2 at least also the containers themselves move and, in this way, the shading effects can change periodically.

In the case of the embodiment shown in FIG. 1 the individual periods also differ at least slightly with respect to the sensor signal shown here, since these are to be allocated in each case to the individual radiation devices which dip into the containers.

In this way, in the two embodiments the periodicity of the sensor signal designates a distribution of the bottles. The references G1 and G2 designate upper and lower threshold values which in this case, as shown in the figures, are adapted to the sensor signal. This adaptation can be carried out in a special learning operation. It is advantageous in this case for the respective radiation values to be received in a manner dependent upon a rotational position of the conveying device and thus for the threshold value patterns G1 and G2 to be selected for example by additions and reductions of 10% in each case with respect to the signal pattern. The radiation values can be received in this case in a manner dependent upon rotary encoder settings of the conveying device. In this case it would be possible for this pattern to be received with a pre-set number of support points. Values between these support points can be determined for example by interpolation. In addition, it would be possible to average the radiation patterns over a multiplicity of revolutions. The formation of sliding averages would also be possible. In addition, however, it would also be possible for the pattern of the threshold values to be indicated or approximated, for example a sine function or a square function or combinations of a multiplicity of functions. In this case it would optionally be possible to dispense with a learning operation.

The measured sensor signal must therefore be present at any time within the tolerance range which is formed between these two threshold values G1 and G2. In the illustration reproduced in FIG. 3b the sensor signal deviates in a range S1 and exceeds the upper threshold value G1. For this reason it is possible to conclude that a specific radiation device is defective. In the case of this exceeding of the signal it would be possible for the plant to be switched off immediately. In this case the sensor signal S1 in a third container leaves the tolerance range, so that it can be concluded that the corresponding radiation device is defective. For monitoring purposes it would also be possible for a multiplicity of periods to be measured or during a multiplicity of complete revolutions of the conveying device. In this way, it would he possible to check whether the signal of the same radiation device in each case leaves the tolerance range several times.

In the case of the design shown in FIG. 3c, it will be seen that the signal which is likewise associated with a specified radiation device in that case fails to reach the lower threshold value G2. In this case too, it can be concluded that the radiation device in question is no longer radiating (sufficiently). It would be possible in this case likewise to switch off the plant and to carry out repair operations on the defective radiation device in question. Within the framework of repair operations radiators recognized as being defective can preferably be brought automatically or independently respectively into a servicing position, for example into the region of a servicing opening.

It would also be possible, however, for the plant to be allowed to continue to operate for a time and for all the containers which have been sterilized by lust this defective radiator to be separated out. It is likewise possible to occupy on the inlet side only the positions which have radiators which are functioning properly.

FIGS. 4a-c show three designs for the arrangements of the radiation detector device. The containers are not indicated in this case. In the situation shown in FIG. 4a the sensor device simultaneously receives a signal from different sources. This leads to a diffuse superimposition which possibly makes it difficult to identify a specified radiation device. In the situation shown in FIG. 4b a housing 62 is provided which has an inlet opening 64. It will be seen that in this case the sensor device detects only the radiation from a single source 4 and the other radiations do not reach the sensor device. In this way, the measured signal is modulated more strongly, and it is thus possible for a specified radiation device which is possibly defective to be allocated clearly at each position.

In the situation shown in FIG. 4c the sensor device no longer detects direct radiation, but only diffuse background by scattered radiation 4 which is to be ascribed to the radiation device 4′.

FIG. 5 is an illustration of a plant according to the invention for the sterilization of containers, in this case of plastics material pre-forms. Here the plastics material pre-forms are conveyed and first sterilized on the outer wall thereof by two sterilization devices 8. The two sterilization devices 8 are arranged in this case on opposite sides of the conveying path and also offset from each other. The reference numbers 42 and 44 designate walls which screen off the conveying path of the containers. These walls can on the one hand bound a clean room in this case, inside which the containers are conveyed, but on the other hand they can also serve for screening off the x-ray radiation which is produced. The reference number 32 designates a transfer star wheel which conveys the containers from the-external sterilization to the internal sterilization.

In this way, the external sterilization of the containers is followed by an internal sterilization, in which case, as shown above, radiation fingers are inserted in each case into the interior of the containers. The reference number 50 refers to the clean room inside which the containers are sterilized.

FIG. 6 is a further illustration to demonstrate the internal, sterilization. In this case reciprocating devices 36 are also shown, which lift the containers 10 and thus cause the radiation fingers 12 to be inserted into the containers.

It is preferable in this case for sensor devices 6 to be positioned in the region of the radiation fingers 12, in a particularly preferred manner at the side and in a particularly preferred manner at a level just below the radiation exit window. The sensor devices then detect the x-ray radiation of the emitters moving past. In terms of safety it is advantageous for a sensor device or a detector based upon a scintillator and a sensor device based upon an ionization chamber to be used.

The first sensor device 6 (on the left in the figure) receives the modulated signal and evaluates whether there has been a departure from the tolerance range. It decides at the start of the processing whether the pre-form or the container respectively will be irradiated properly.

The second sensor device (on the right in the figure) arranged downstream with respect to the first sensor device can confirm the results of the first sensor device 6 and additionally detects whether one of the radiators has gone wrong during the processing and whether it becomes necessary for the container 10 in question as the pre-forms in question respectively to be separated out It is assumed in this case that individual radiators between these two detectors were functioning in a manner in accordance with their intended use. A fault on account of an arc in an emitter is not ascertained in this way, this error being detected in the generator of the radiation device.

FIG. 7 is an enlarged illustration to demonstrate the external sterilization of the containers. Two sensor devices 6 (shown only diagrammatically) are arranged in the region of the two surface radiators 8 here. In this case the sensor devices here are arranged below the conveying path of the containers 10.

The positioning of these sensor devices 6 could be carried out in such a way that the two sensor devices or detectors respectively detect mainly one radiation source in each case. It is particularly preferred for them to be shaded (for example by means of a screen) in such a way that they receive a strong signal only when a pre-form is directly passing. They should also, however, detect scattered radiation of the adjacent pre-forms and/or of the adjacent emitter. The two detectors should thus be subject to a similar signal.

This ensures that a fault can be assigned to the pre-form or station in question. Nevertheless, the two detectors also detect a background radiation if, at this time, a pre-form is not above it. The basic principle of the emitter is thus confirmed.

The Applicants reserve the right to claim individual features, a multiplicity of the features or all the features disclosed as being essential to the invention, in particular insofar as they are novel either individually or in combination as compared with the prior art.

### LIST OF REFERENCES

1 apparatus

2 conveying device/conveying wheel

4 radiation device for internal sterilization

4′ radiation device

6 radiation detector device/sensor device

8 radiation device for external sterilization

10 containers

12 radiation fingers

16 holding devices

20 control device

22 supply wheel

24 removal wheel

25 memory device

26 rotary encoder

28 comparator device

32 transfer star wheel

36 reciprocating device.

42, 44 wall

50 clean room

62 housing

64 inlet opening

G1, G2 threshold values

P conveying path

S, S1 sensor signal

L longitudinal direction of the containers

1. A method of operating an apparatus for the sterilization of containers, comprising: the containers are conveyed along a pre-set conveying path by means of a conveying device and during this conveying at least one wall of the containers is irradiated with a radiation—and in particular a charge carrier radiation—by means of at least one first radiation device; wherein, in addition, a sensor device is provided which detects charge carrier radiation and/or radiation produced by the charge carriers and which is arranged in such a way that the radiation striking the sensor device is subjected to fluctuations on account of the movement of the containers along the conveying path; and this sensor device detects at least one pattern characteristic of the radiation; wherein at least one first threshold value is defined and a comparison is made between this threshold value and the pattern and wherein the threshold value has a value which is variable in terms of time and/or location.

2. The method according to claim 1, wherein the threshold value is adapted to the time and/or location pattern characteristic of the radiation.

3. The method according to claim 1, wherein the sensor device is arranged in such a way that at least portions of the containers are conveyed between the radiation device and the sensor device.

4. The method according to claim 1, wherein by the comparison between the threshold value and the pattern it is determined whether the irradiation of the containers meets pre-set criteria.

5. The method according to claim 1, wherein at least one second threshold value is defined and a comparison is made between this second threshold value and the pattern.

6. The method according to claim 1, wherein the first radiation device is moved with the containers.

7. The method according to claim 1, wherein at least one portion of the radiation device is inserted into an interior of the containers.

8. The method according to claim 1, wherein a defined radiation device is identified from a comparison between the pattern and the threshold value.

9. An apparatus for the sterilization of containers, comprising: a conveying device which conveys the containers along a pre-set conveying path; with at least one first radiation device which acts upon at least one wall of the containers with a radiation, and in particular a charge carrier radiation; with a sensor device which detects charge carrier radiation and/or radiation produced by the charge carriers and which is arranged in such a way that the radiation striking the sensor device is subjected to fluctuations on account of the movement of the containers along the conveying path; wherein this sensor device detects at least one value pattern characteristic of the radiation; and with a comparator device which compares the characteristic value pattern with at least one first threshold value, wherein the threshold value has a variable value.

10. The apparatus according to claim 9, wherein the apparatus has a plurality of radiation devices which in each case irradiate different containers.

11. The apparatus according to claim 9, wherein the apparatus has a position detection device which detects a position of the conveying device.
